(19) United States
(12) Patent Application Publication
(10) Pub. No.: US 2023/0410497 A1
(43) Pub. Date: Dec. 21, 2023

(54) MEDICAL IMAGE PROCESSING APPARATUS AND MEDICAL IMAGE PROCESSING METHOD

(71) Applicant: CANON MEDICAL SYSTEMS CORPORATION, Tochigi (JP)

(72) Inventors: Xu YANG, Beijing (CN); Fanjie MENG, Beijing (CN); Tianhong LI, Beijing (CN); Xinyao LI, Beijing (CN)

(73) Assignee: CANON MEDICAL SYSTEMS CORPORATION, Tochigi (JP)

(21) Appl. No.: 18/334,779

(22) Filed: Jun. 14, 2023

(30) Foreign Application Priority Data
Jun. 15, 2022 (CN) 202210676793.3

(51) Int. Cl. G06V 10/94; G06V 20/70; G06V 10/40; G16H 30/40

(52) U.S. Cl. CPC G06V 10/945; G06V 20/70; G06V 10/40; G16H 30/40; G06V 2201/03

(57) ABSTRACT

A medical image processing apparatus according to an embodiment includes processing circuitry. The processing circuitry is configured to obtain a medical image subject to a labeling process. The processing circuitry is configured to receive a labeling step in a labeling task performed on the medical image. The processing circuitry is configured, while the labeling step in the labeling task is received, to analyze a local characteristic of a target structure serving as a labeling target in the medical image. The processing circuitry is configured to generate a usable tool set corresponding to the labeling task performed on the medical image, on the basis of the local characteristic.

### CROSS-REFERENCE TO RELATED APPLICATIONS

This application is based upon and claims the benefit of priority from Chinese Patent Application No. 202210676793.3, filed on Jun. 15, 2022, the entire contents of all of which are incorporated herein by reference.

### FIELD

Embodiments described herein relate generally to a medical image processing apparatus and a medical image processing method.

### BACKGROUND

Users who are image interpreting doctors have a medical image displayed on a display at the time of interpreting the medical image, which may be an X-ray image, a Computed Tomography (CT) image, an ultrasound image, or the like. Further, by using various types of tools provided as applications, the users perform labeling processes (which may be referred to as “annotation”) such as segmentation, classification, and detection, so as to obtain an image of a desired region or a labeled medical image.

Software for labeling medical images offers a plurality of types of tools. When using such software, it is difficult for users to quickly find an optimal tool for a labeling task at present, because there are many types of tools. Further, when having the same labeling task, a user may be required to label a large amount of data and to manually perform a number of duplicate operations such as display adjustments. For these reasons, as for the current tendencies related to using labeling tools, labeling takes a long time and has low efficiency.

To cope with the problems described above, a method has been proposed by which a workflow including tools that need to be used for a specific segmentation task is designated, so that a wizard instructs user operations.

However, the abovementioned method is used only for the specific segmentation task and is not able to meet the needs in other labeling tasks. Further, because different wizards need to be developed for different segmentation tasks, it is not possible to support users' needs in a timely manner.

### BRIEF DESCRIPTION OF THE DRAWINGS

FIG. 1 is a block diagram illustrating a functional configuration of a medical image processing apparatus according to a first embodiment;

FIG. 2A is a schematic drawing illustrating an example of a labeling assistance information table;

FIG. 2B is a schematic drawing illustrating an example of an anatomical similarity table;

FIG. 3A is a schematic drawing illustrating a gradation histogram used at the time of judging applicability of a Threshold tool;

FIG. 3B is a schematic drawing illustrating another gradation histogram used at the time of judging the applicability of the Threshold tool;

FIG. 4A is a schematic drawing for explaining a judgment condition at the time of judging the applicability of the Threshold tool;

FIG. 4B is a schematic drawing for explaining another judgment condition at the time of judging the applicability of the Threshold tool;

FIG. 5 is a flowchart for judging the applicability of the Threshold tool;

FIG. 6 is a flowchart for judging applicability of a Livewire tool;

FIG. 7 is a schematic drawing illustrating an example of a tool management table;

FIG. 8 is a flowchart in which a workflow generating function of the medical image processing apparatus according to the first embodiment records user operations and image states;

FIG. 9 is a schematic drawing illustrating an exemplary configuration of a workflow;

FIG. 10 is a schematic drawing illustrating an example to which a tool set is applied;

FIG. 11 is a schematic drawing illustrating a display example of when a display part of the workflow is applied;

FIG. 12 is a schematic drawing illustrating a display example of when a labeling part of the workflow is applied;

FIG. 13 is a flowchart illustrating a procedure in a process (a medical image processing method) performed by the medical image processing apparatus according to the first embodiment;

FIG. 14 is a block diagram illustrating a functional configuration of a medical image processing apparatus according to a second embodiment;

FIG. 15 is a schematic drawing illustrating a labeled result from a lung part segmentation labeling task presented as an example for explaining a tool set optimizing process;

FIG. 16 is a flowchart for judging applicability of an automatic interpolation tool in the tool set optimizing process;

FIG. 17A is a schematic drawing for explaining an example of a workflow optimizing process;

FIG. 17B is another schematic drawing for explaining the example of the workflow optimizing process;

FIG. 18 is a schematic drawing for explaining another example of the workflow optimizing process;

FIG. 19 is a schematic drawing for explaining yet another example of the workflow optimizing process; and

FIG. 20 is a flowchart illustrating a procedure in a process (a medical image processing method) performed by the medical image processing apparatus according to the second embodiment.

### DETAILED DESCRIPTION

A medical image processing apparatus according to an embodiment of the present disclosure includes processing circuitry. The processing circuitry is configured to obtain a medical image subject to a labeling process. The processing circuitry is configured to receive a labeling step in a labeling task performed on the medical image. The processing circuitry is configured, while the labeling step in the labeling task is received, to analyze a local characteristic of a target structure serving as a labeling target in the medical image. The processing circuitry is configured to generate a usable tool set corresponding to the labeling task performed on the medical image, on the basis of the local characteristic.

Exemplary embodiments of a medical image processing apparatus and a medical image processing method will be explained in detail below, with reference to the accompanying drawings.

A medical image processing apparatus according to an embodiment of the present disclosure is structured with a plurality of functional modules and is realized as a result of a processor executing the functional modules of the medical image processing apparatus stored in a memory, by installing the functional modules as software into a machine such as an independent computer having a Central Processing Unit (CPU) and the memory or installing the functional modules into a plurality of machines in a distributed manner.

Alternatively, the medical image processing apparatus may be realized in the form of hardware, as circuitry capable of executing the functions of the apparatus. Further, the circuitry realizing the medical image processing apparatus is capable of transmitting and receiving data and acquiring data via a network such as the Internet. Furthermore, the medical image processing apparatus according to the present embodiment may directly be provided in a medical image acquiring apparatus such as a CT apparatus or a magnetic resonance imaging apparatus, as a part of the medical image acquiring apparatus.

### First Embodiment

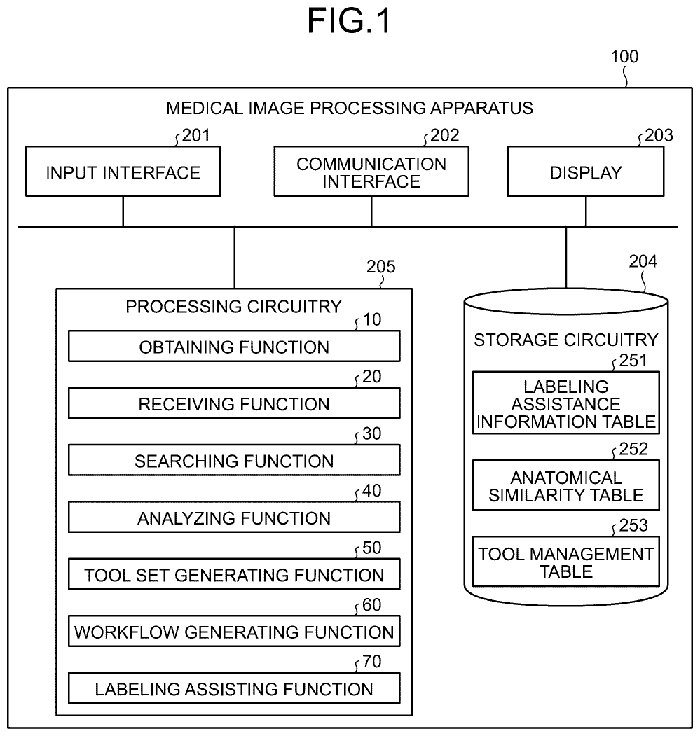

To begin with, a first embodiment will be explained, with reference to FIGS. 1 to 13.

FIG. 1 is a block diagram illustrating a functional configuration of a medical image processing apparatus 100 according to the first embodiment. As illustrated in FIG. 1, the medical image processing apparatus 100 includes an input interface 201, a communication interface 202, a display 203, storage circuitry 204, and processing circuitry 205. In the present embodiment, the medical image processing apparatus 100 is an apparatus used by a user such as a medical doctor (e.g., an image interpreting doctor) for observing and interpreting medical images. The medical image processing apparatus 100 may be a user terminal.

The input interface 201 is realized by using a trackball, a switch button, a mouse, a keyboard, a touchpad on which an input operation can be performed by touching an operation surface thereof, a touch screen in which a display screen and a touchpad are integrally formed, contactless input circuitry using an optical sensor, audio input circuitry, and/or the like which are used for establishing various settings or the like. The input interface 201 is connected to the processing circuitry 205 and is configured to convert input operations received from the user such as a medical doctor into electrical signals and to output the electrical signals to the processing circuitry 205. Although being provided within the medical image processing apparatus 100 in FIG. 1, the input interface 201 may be provided on the outside thereof.

The communication interface 202 is a Network Interface Card (NIC) or the like and is configured to communicate with other apparatuses. For example, the communication interface 202 is connected to the processing circuitry 205 and is configured to acquire medical images from an ultrasound diagnosis apparatus serving as an ultrasound system or other modalities besides the ultrasound system such as an X-ray Computed Tomography (CT) apparatus or a Magnetic Resonance Imaging (MRI) apparatus and configured to output the acquired images to the processing circuitry 205.

The display 203 is connected to the processing circuitry 205 and is configured to display various types of information and various types of images output from the processing circuitry 205. For example, the display 203 is realized by using a liquid crystal monitor, a Cathode Ray Tube (CRT) monitor, a touch panel, or the like. For example, the display 203 is configured to display a Graphical User Interface (GUI) for receiving instructions from the user, various types of display images, and various processing results obtained by the processing circuitry 205. Although being provided within the medical image processing apparatus 100 in FIG. 1, the display 203 may be provided on the outside thereof.

The storage circuitry 204 is connected to the processing circuitry 205 and is configured to store therein various types of data. More specifically, the storage circuitry 204 is configured to store therein, at least, various types of medical images for an image registration purpose and fusion images or the like obtained after the registration. For example, the storage circuitry 204 is realized by using a semiconductor memory element such as a Random Access Memory (RAM) or a flash memory, or a hard disk, an optical disk, or the like. Further, the storage circuitry 204 is configured to store therein programs corresponding to processing functions executed by the processing circuitry 205. Although being provided within the medical image processing apparatus 100 in FIG. 1, the storage circuitry 204 may be provided on the outside thereof.

Further, the storage circuitry 204 has stored therein a labeling assistance information table 251, an anatomical similarity table 252, and a tool management table 253. The information stored in the labeling assistance information table 251, the anatomical similarity table 252, and the tool management table 253 will be explained later.

For example, the processing circuitry 205 is realized by using a processor. As illustrated in FIG. 1, the processing circuitry 205 includes an obtaining function 10, a receiving function 20, a searching function 30, an analyzing function 40, a tool set generating function 50, a workflow generating function 60, and a labeling assisting function 70. In this situation, processing functions executed by the constituent elements of the processing circuitry 205 illustrated in FIG. 1, namely, the obtaining function 10, the receiving function 20, the searching function 30, the analyzing function 40, the tool set generating function 50, the workflow generating function 60, and the labeling assisting function 70, are recorded in the storage circuitry 204 of the medical image processing apparatus 100 in the form of computer-executable programs, for example. The processing circuitry 205 is a processor configured to realize the processing functions corresponding to the programs, by reading and executing the programs from the storage circuitry 204. In other words, the processing circuitry 205 that has read the programs has the functions illustrated within the processing circuitry 205 in FIG. 1.

The term “processor” used in the above explanations denotes, for example, a Central Processing Unit (CPU), a Graphics Processing Unit (GPU), or circuitry such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)). When the processor is a CPU, for example, the processor is configured to realize the functions by reading and executing the programs saved in the storage circuitry 204. In contrast, when the processor is an ASIC, for example, instead of having the programs saved in the storage circuitry 204, the programs are directly incorporated in the circuitry of the processor. Further, the processors of the present embodiment do not each necessarily have to be structured as a single piece of circuitry. It is also acceptable to structure one processor by combining together a plurality of pieces of independent circuitry so as to realize the functions thereof. Furthermore, it is also acceptable to integrate two or more of the constituent elements in FIG. 1 into a processor, so as to realize the functions thereof.

Next, details of processes performed by the obtaining function 10, the receiving function 20, the searching function 30, the analyzing function 40, the tool set generating function 50, the workflow generating function 60, and the labeling assisting function 70 executed by the processing circuitry 205 will be explained.

The obtaining function 10 is configured to obtain a medical image that was acquired by scanning an examined subject (hereinafter, “patient”) and needs to be labeled, from a database in a medical facility such as a hospital or an image acquiring apparatus such as an ultrasound diagnosis apparatus, an X-ray radiating apparatus, or the like. The medical image is subject to a labeling process performed with any of various types of labeling tools. In other words, the obtaining function 10 is configured to obtain the medical image subject to the labeling process. The obtaining function 10 is an example of an “obtaining unit”.

The receiving function 20 is configured to receive labeling steps in a labeling task performed on the medical image obtained by the obtaining function 10. The receiving function 20 is an example of a “receiving unit”.

The labeling process denotes a step of performing a process such as segmentation, classification, detection, or the like on the medical image and adding a symbol indicating labeling information to the medical image. The labeling process may be called annotation. By using a region focused on in the labeling process as a region of interest, and in the present embodiment, as a target structure serving as a labeling target, it is possible to emphasize an important region to be labeled so as to be used in model training of Artificial Intelligence (AI). Further, the target structure is a structure set by a user or the like in accordance with a purpose of the model training. For example, when an AI model for segmenting the liver is trained, the liver is set as a target structure. When an AI model for classifying benignity/malignancy of a tumor is trained, a tumor region is set as a target structure. When an AI model for detecting lung nodules is trained, a lung nodule is set as a target structure. The target structure may be set automatically according to industrial standards in the relevant field. For example, it is possible to determine a target structure by referring to the pharmaceutical industry standards “Artificial Intelligence Medical Device Quality Requirements and Evaluation, Part 1”.

In the present embodiment, an example of a labeling task will be explained in which the user such as a medical doctor performs segmentation on a target structure in a medical image, via an input/output apparatus such as a human machine interface.

Steps in the labeling process include, generally, the user's defining a labeling task, a display step of adjusting a display state, and a step of labeling an image by using a labeling tool. The receiving function 20 is configured, via an input/output apparatus such as a human machine interface, to receive data generated in the labeling steps and various types of processes performed on the medical image. For example, via an interface displayed on a display, the receiving function 20 is configured to receive the labeling task defined by the user, loading of data, and the labeling process performed by the user by using the labeling tool. The labeling task is defined via an input of the user and prescribes relevant information for identifying the task, such as a labeling type (segmentation, etc.), a target view (two-dimensional, etc.), a target structure (the liver, etc.) to be segmented, and a mechanism used for the acquisition (CT, etc.), for example.

On the basis of the labeling task received by the receiving function 20, the searching function 30 is configured to conduct a search to determine whether or not a usable tool set corresponding to the obtained medical image labeling task is present. Further, the searching function 30 is configured to conduct a search to determine whether or not an existing workflow corresponding to the medical image labeling task is present. The searching function 30 is an example of a “searching unit”.

The usable tool set (which may simply be referred to as “tool set”) is a set of tools that are usable in the labeling steps. The tools are represented by software applications that are provided by one or more software venders and assist the labeling process performed by the user. Details of the usable tool set will be explained later.

The existing workflow (which may simply be referred to as “workflow”) is a labeling flow prescribing the labeling steps. The medical image processing apparatus 100 is configured to save a plurality of tool sets and a plurality of workflows in advance so that, at the time of a labeling process, it is possible to search for and to use a tool set and a workflow suitable for a labeling task. Details of the workflow will be explained later.

The searching function 30 is configured to search for the tool set and the workflow, by referring to the labeling assistance information table 251 which is stored in advance and in which labeling tasks are kept in correspondence with either identifiers of tool sets or identifiers of workflows.

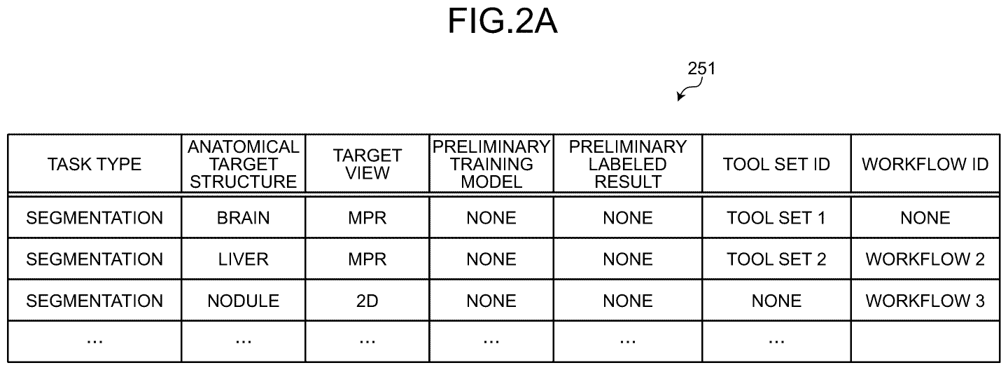

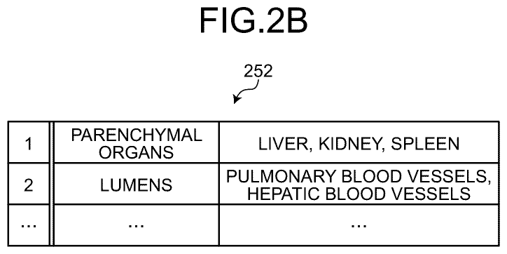

FIG. 2A is a schematic drawing illustrating an example of the labeling assistance information table 251. As illustrated in FIG. 2A, the labeling assistance information table 251 has stored therein task types, anatomical target structures, target views, tool set IDs, workflow IDs, and other information (e.g., a preliminary training model, preliminary labeled results) that are kept in correspondence with one another. When the user has defined a labeling task, the searching function 30 is configured to search in the labeling assistance information table 251 on the basis of at least one of a task type input by the user and task information such as an anatomical target structure, so as to judge whether or not a usable tool set and an existing workflow are present. For example, let us discuss an example in which the task type is segmentation, whereas the target structure is the liver. In that situation, the searching function 30 is configured to conduct a search by using the labeling assistance information table 251 while using segmentation and the liver as keywords. In the example illustrated in FIG. 2A, the searching function 30 finds the tool set ID “TOOL SET 2” and the workflow ID “WORKFLOW 2” corresponding to the second entry and determines that a usable tool set and an existing workflow for the abovementioned task are present.

Further, the searching function 30 is also capable of conducting a search on the basis of at least one piece of information in the labeling task and is thus capable, even when there is a difference in the other information (e.g., when only one information item is different), of adopting a usable tool set and an existing workflow found in the search while ignoring the difference. Furthermore, when the searching function 30 is unable to find in the search a usable tool set and an existing workflow corresponding to a keyword from the labeling assistance information table 251, it is possible to conduct a search by using another keyword similar to the prescribed keyword. For example, when a target structure is used as a keyword, it is possible to conduct a search by using another target structure in accordance with a similar organ listed in the anatomical similarity table 252.

FIG. 2B is a schematic drawing illustrating an example of the anatomical similarity table 252. As illustrated in FIG. 2B, “LIVER”, “KIDNEY”, and “SPLEEN” all belong to parenchymal organs, and let us assume that the labeling task is a task for segmenting a kidney. In this situation, to begin with, the searching function 30 conducts a search in the labeling assistance information table 251 in FIG. 2A. In this situation, when being unable to find a corresponding entry for the kidney segmentation, the searching function 30 refers to the anatomical similarity table 252 in FIG. 2B, determines that the liver and the spleen are structures similar to the kidneys, and uses “liver” and “spleen” as keywords. Accordingly, the searching function 30 conducts a search in the labeling assistance information table 251 in FIG. 2A, by using “liver” and “spleen” as keywords. In the example illustrated in FIG. 2A, the searching function 30 finds the tool set ID “TOOL SET 2” and the workflow ID “WORKFLOW 2” corresponding to the second entry and determines that a usable tool set and an existing workflow for the abovementioned task are present.

In other examples, when the medical image processing apparatus 100 has not stored therein the information about the usable tool sets and the existing workflows or when the medical image processing apparatus 100 does not use an existing usable tool set and an existing workflow, the searching function 30 may be omitted.

While the receiving function 20 is receiving the labeling steps in the labeling task, the analyzing function 40 is configured to analyze a local characteristic of the target structure serving as a labeling target in the medical image. Accordingly, the tool set generating function 50 is configured to generate a usable tool set corresponding to the medical image labeling task, on the basis of the local characteristic analyzed by the analyzing function 40. The analyzing function 40 is an example of an “analyzing unit”. The tool set generating function 50 is an example of a “tool set generating unit”.

More specifically, after the labeling process is started, the analyzing function 40 is configured to analyze the local characteristic of the target structure, on the basis of a partial labeling process occurring at a different stage in the labeling steps. For example, when the analyzing function 40 starts segmenting a pulmonary blood vessel as a labeling process, segmentation performed on a part of the pulmonary blood vessel is referred to as a “partial labeling process”. Further, the analyzing function 40 is configured to analyze the local characteristic of the target structure on the basis of the partial labeling process performed on the part of the pulmonary blood vessel. The local characteristic is used for judging whether or not a certain tool is suitable for the labeling task at this time. Types of local characteristics that require analyses may be set in accordance with affecting factors of candidate tools. Alternatively, a plurality of types of local characteristics may be set in advance, so as to be used for judging a plurality of tools.

Further, on the basis of the local characteristic analyzed by the analyzing function 40, the tool set generating function 50 is configured to generate the usable tool set that corresponds to the medical image labeling task and is structured with a plurality of tools. For example, on the basis of the local characteristic, the tool set generating function 50 is configured to sequentially judge whether or not each of all the candidate tools usable or understandable for the user is suitable for the medical image labeling task. After that, upon determining that one or more of the tools is determined to be usable for the medical image labeling task, the tool set generating function 50 is configured to add the one or more tools to the tool set.

In this manner, in the present embodiment, to allow the user to select a tool, it is possible to add various types of tools recommended to be used in the labeling steps, to the usable tool set. The tools used in the labeling steps are software applications that are provided by one or more software venders and assist the user in performing the labeling process. Generally speaking, a plurality of mutually-different tools need to be used in a labeling process at a time. When tools are categorized according to purposes, a usable tool set includes: display tools realized with applications for displaying images; a preliminary labeling tool for performing a preliminary labeling process; and labeling tools for performing labeling processes. In this situation, it would be difficult for users to select a labeling tool as a candidate tool. To cope with this situation, in the present embodiment, an example of a tool set structured with a plurality of labeling tools will be explained. In other words, in the present embodiment, the example will be explained in which the usable tool set is a tool set including the plurality of labeling tools.

For example, an example of a task will be explained in which, while the candidate tool is a Threshold (gradation threshold value division) tool, a pulmonary blood vessel is to be segmented.

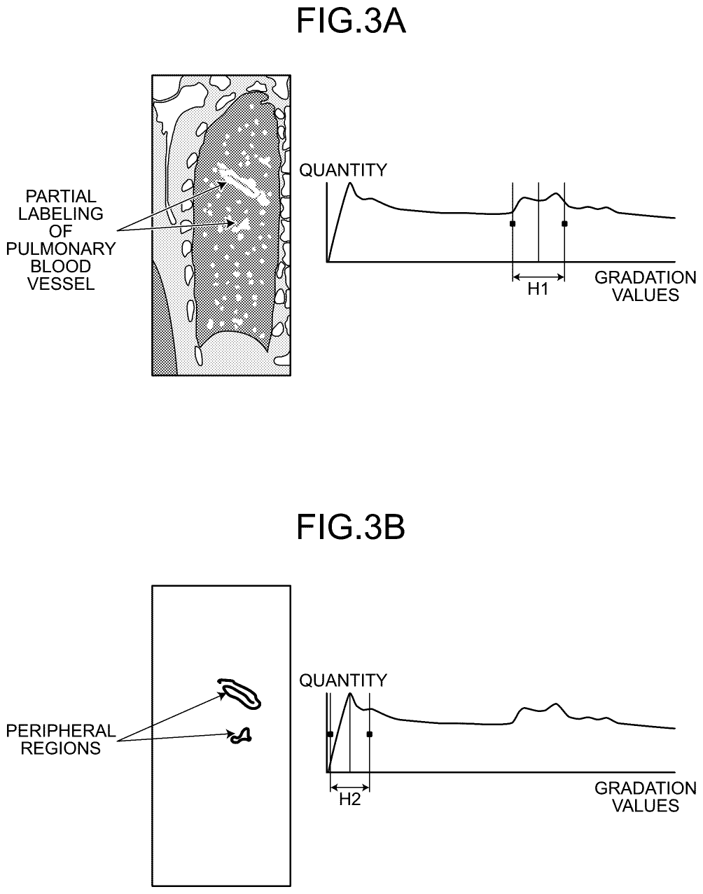

To begin with, when the user sets a labeling task during the labeling steps and invokes data, the analyzing function 40 causes a display to display a medical image of a lung part. Subsequently, after the analyzing function 40 adjusts the display state by using a display tool, the user (e.g., a medical doctor) labels a lung part image displayed on a display screen and blood vessel parts in a slice image and sequentially draws each of the blood vessel parts. In the present embodiment, the labeling tool used for performing the partial labeling process is not limited. For example, in the image on the left-hand side of FIG. 3A, the white regions indicated by the arrows are results of a partial labeling process (in two locations) performed by the user on a pulmonary blood vessel in the medical image. Let us assume that a part of the blood vessel parts has been drawn (i.e., the labeling task is not completed). In this situation, as illustrated in FIGS. 3A and 3B, the analyzing function 40 extracts a gradation value range H1 of the labeled blood vessel parts and a gradation value range H2 of peripheral regions of the blood vessel parts, from a gradation histogram of the medical image.

For example, presented on the right-hand side of FIG. 3A is a gradation histogram of the lung part image on the left-hand side of FIG. 3A. In this situation, on the right-hand side of FIG. 3A, the horizontal axis expresses gradation values of the pixels, whereas the vertical axis expresses the quantity of the pixels having mutually the same gradation value. The pixel value range within the gradation value range H1 is a pixel value range of the labeled blood vessel parts. Further, presented on the left-hand side of FIG. 3B are the peripheral regions of the labeled blood vessel parts. On the left-hand side of FIG. 3B, the peripheral regions are displayed as black regions indicated by the arrows. Presented on the right-hand side of FIG. 3B is the gradation value range H2 in the same gradation histogram as the gradation histogram on the right-hand side of FIG. 3A. On the right-hand side of FIG. 3B, the horizontal axis expresses gradation values of the pixels, whereas the vertical axis expresses the quantity of the pixels having mutually the same gradation value. The pixel value range within the gradation value range H2 is a pixel value range of the peripheral regions of the labeled blood vessel parts.

In the manner described above, on the basis of the gradation histogram of the medical image and the partial labeling process, the analyzing function 40 obtains the gradation value range H1 of the blood vessel parts and the gradation value range H2 of the peripheral regions of the blood vessel parts, as local characteristics. Accordingly, the tool set generating function 50 assigns the local characteristics as a judgment condition of the Threshold tool and judges whether or not the Threshold tool is suitable for the use.

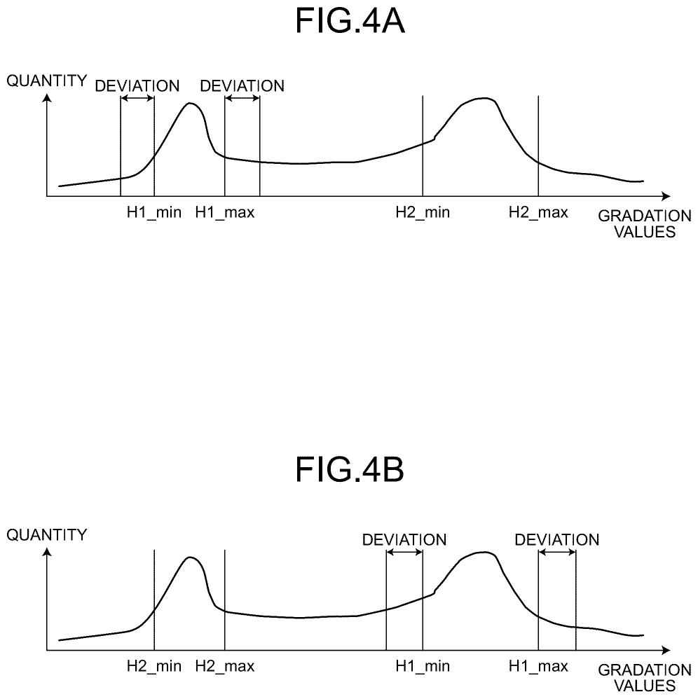

FIGS. 4A and 4B respectively present two judgment conditions satisfying applicability of the Threshold tool. In FIGS. 4A and 4B, the horizontal axis expresses gradation values of pixels, whereas the vertical axis expresses the quantity of the pixels having mutually the same gradation values. For example, upon determining that a judgment condition “H2_min>H1_max+a deviation” is satisfied as illustrated in FIG. 4A, the tool set generating function 50 determines that the Threshold tool is suitable for the use. In another example, upon determining that another judgment condition “H2_max<H1_min−the deviation” is satisfied as illustrated in FIG. 4B, the tool set generating function 50 determines that the Threshold tool is suitable for the use. In this situation, H1_min and H1_max denote a minimum value and a maximum value of H1, respectively. H2_min and H2_max denote a minimum value and a maximum value of H2, respectively. The “deviation” is a fixed deviation value added to the threshold value process.

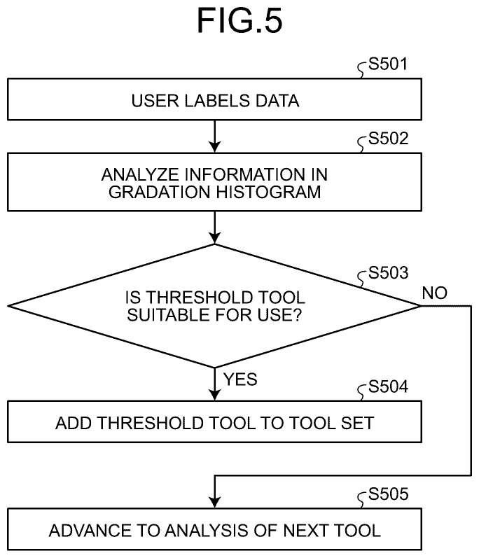

FIG. 5 is a flowchart for judging the applicability of the Threshold tool. As illustrated in FIG. 5, at the step (step S501) in which the user labels data, the analyzing function 40 analyzes the information in the gradation histogram after the partial labeling process is performed (step S502). In that situation, at step S502, the analyzing function 40 extracts the gradation value range H1 of the labeled blood vessel parts and the gradation value range H2 of the peripheral regions of the blood vessel parts, as the local characteristics. In this situation, the timing for executing the partial labeling process is set in advance. For example, the analyzing function 40 may be configured to perform the analysis when the length or the area of patterns in the received labeling process has reached a certain level, or the analyzing function 40 may be configured to perform the analysis in accordance with a successively-labeled quantity.

Subsequently, at step S503, the tool set generating function 50 judges whether or not the gradation value ranges H1 and H2 satisfy the predetermined judgment conditions for the Threshold tool. In this situation, when any of the judgment conditions for the Threshold tool is satisfied (step S503: Yes), the tool set generating function 50 adds the Threshold tool to the tool set (step S504). On the contrary, when none of the judgment conditions for the Threshold tool is satisfied (step S503: No), the tool set generating function 50 judges the next candidate tool (step S505).

Next, another example of a labeling task will be explained in which, while the candidate tool is a Livewire (magnet selection) tool, the lungs are to be segmented, for instance. In this situation, an image dividing method for extracting a contour of a region of interest is called a Livewire method. According to the Livewire method, two points, namely a start point and an end point, are given so as to extract the contour of the region of interest between the two given points. A Livewire tool is a tool implementing the Livewire method.

For example, at the time of segmenting the lungs, in many situations, a plurality of slice images are selected from the entire lungs, so as to label the plurality of slice images with a dividing line all at once, so as to subsequently perform collective processes such as an interpolating process. For this reason, in the present embodiment, when a first slice image is labeled with a closed region (either the left lung or the right lung), i.e., when a partial labeling process has been performed thereon, it is judged whether or not the Livewire tool is suitable for the use.

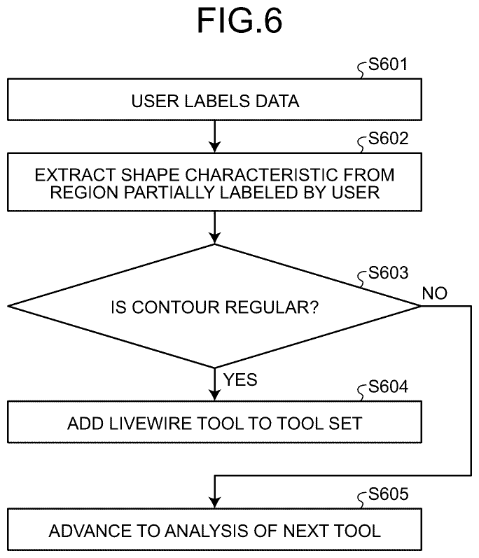

FIG. 6 is a flowchart for judging the applicability of the Livewire tool. As illustrated in FIG. 6, during the step (step S601) at which the user labels data, the analyzing function 40 extracts a shape characteristic of the local region partially labeled by the user, as a local characteristic (step S602).

Subsequently, at step S603, the tool set generating function 50 judges whether or not the contour of the partially-labeled local region is regular, on the basis of the extracted shape characteristic. In this situation, as for a criterion for judging whether or not the contour is regular, it is possible to adopt any of various types of judgment criteria based on conventional techniques. Upon determining that the contour is regular (step S603: Yes), the tool set generating function 50 adds the Livewire tool to the tool set (step S604). On the contrary, upon determining that the contour is not regular (step S603: No), the tool set generating function 50 judges the next candidate tool (step S605).

As explained above, in the present embodiment, the plurality of candidate tools are judged for suitability for the use, so as to add one or more tools determined to be suitable for the use to the tool set and to form the tool set corresponding to the labeling task of the relevant classification type.

Further, the medical image processing apparatus 100 according to the first embodiment is also capable of generating a workflow. Returning to the description of FIG. 1, the workflow generating function 60 is configured, during the medical image labeling steps, to record the labeling steps in the labeling task received by the receiving function 20 and to generate a workflow indicating the medical image labeling steps. The workflow generating function 60 is an example of a “workflow generating unit”.

More specifically, the workflow generating function 60 is capable of recording, in the workflow, details of user actions, use of tools, results of the use, and the like. Generally speaking, the labeling steps are steps of sequentially using various types of tools on a medical image. Accordingly, the workflow generating function 60 is configured to classify tools as described below by referring to the tool management table 253 identifying the tools in advance, to further set a type number for each type, and to set a tool number for each tool. The workflow generating function 60 is configured to identify each of the tools by using a combination of a type number and a tool number.

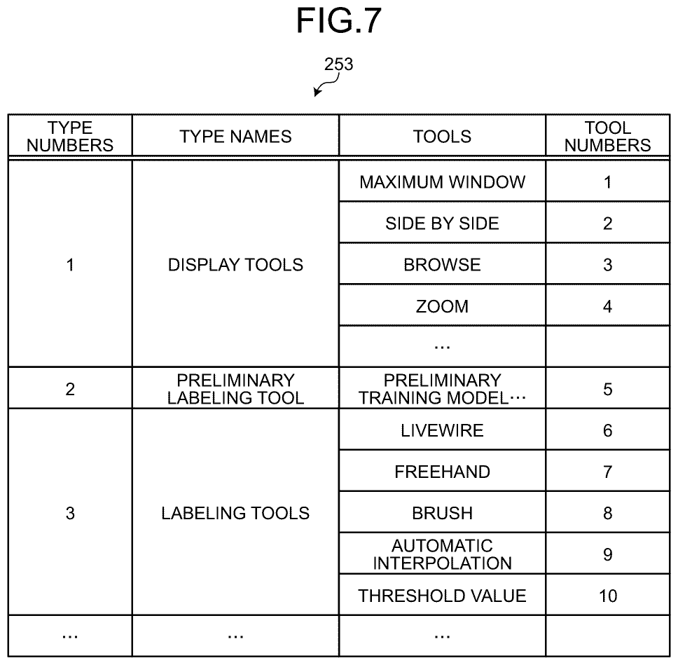

FIG. 7 is a schematic drawing illustrating an example of the tool management table 253. For example, as illustrated in FIG. 7, the tools are classified into three types of tools such as “display tools”, “preliminary labeling tool”, and “labeling tools”, while each of the tools corresponds to one processing program (processing process). To the three types of tool classifications, type numbers 1 to 3 are assigned, respectively. In the present example, included under the type name “display tools” are tools such as: “maximum window” to display a screen with a maximized window (screen) size; “side by side” to display screens next to each other; “browse” to display data and/or information on a screen so as to be easily skimmed through; and “zoom” to display data and/or information on a screen in an enlarged or reduced manner. Included under the type name “preliminary labeling tool” is a tool such as a preliminary training model. Included under the type name “labeling tools” are tools such as: “Livewire (magnet selection)” which is a tool for labeling using the Livewire method; “freehand” which is a tool for freehand labeling by a user; “brush” which is a tool for labeling by a user using a brush; “automatic interpolation” which is a tool for labeling using an input history of a user; and “threshold value” which is the abovementioned Threshold (gradation threshold value division) tool. To these tools, tool numbers 1 to 10 are assigned, respectively. By using the tool management table 253, it is possible to identify the tools on the basis of the tool numbers and to identify the type to which each tool belongs on the basis of the type numbers. It is thus possible to adopt different processing schemes for different types of tools.

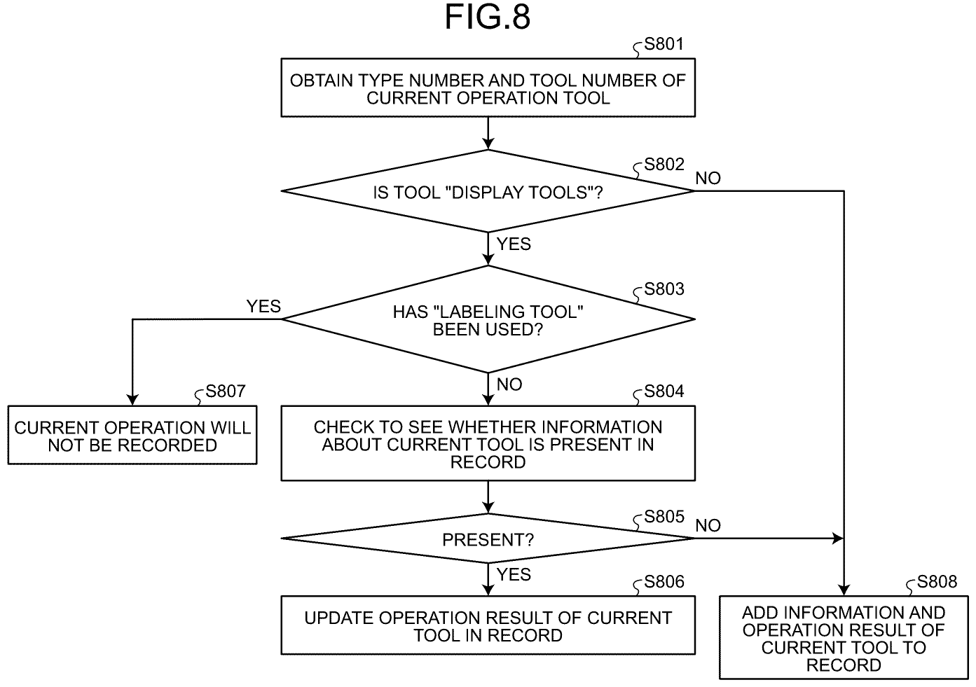

During the labeling steps, the workflow generating function 60 is configured to obtain the type numbers and the tool numbers of the tools used in the labeling steps, to sequentially record the use of the tools and results of the use, and to form a workflow. In an example, the workflow generating function 60 may simplify the recording of the workflow, by not using a number of tools or not using duplicate tools according to prescribed rules. For example, after a user has labeled a medical image of which the display state has been adjusted by using a labeling tool for the first time, a further adjustment operation on the display state is considered to be of little reproduction value. Thus, as illustrated in FIG. 8, it is acceptable to record only the use of display tools before the first-time use of the labeling tool.

FIG. 8 illustrates an example in which the workflow generating function 60 of the medical image processing apparatus 100 according to the first embodiment records user operations and image states. After the user defines a labeling task and loads a medical image on a display screen of a display, the workflow generating function 60 is configured to start recording the tools used for the operations occurring in the labeling task. FIG. 8 illustrates a flow in which an operation tool is recorded upon the occurrence of an operation each time. In the present example, the operations include artificial operations performed by the user and operations automatically performed by mechanisms.

To begin with, upon the occurrence of an operation at a time, the workflow generating function 60 obtains the type number and the tool number of a current operation tool (step S801) and judges whether or not the currently-used operation tool belongs to the “display tools” listed in FIG. 7 on the basis of the type number (step S802). When the operation tool does not belong to the “display tools” (step S802: No), the workflow generating function 60 adds information about the current operation tool and an operation result to the record of the workflow (step S808).

On the contrary, upon determining that the current operation tool belongs to the “display tools” (step S802: Yes), the workflow generating function 60 further judges whether or not a labeling tool was used, i.e., whether or not the recorded workflow includes a record of a labeling tool (step S803). Upon determining that a labeling tool was used (step S803: Yes), the workflow generating function 60 does not record the current operation (step S807).

On the contrary, upon determining that no labeling tool was used (step S803: No), the workflow generating function 60 checks to see whether or not the record of the workflow includes information about the current tool (step S804). Upon determining that the information about the current tool is present (step S805: Yes), the workflow generating function 60 updates the operation result of the current tool being recorded (step S806).

On the contrary, upon determining that no information about the current tool is present (step S805: No), the workflow generating function 60 adds information about the current operation tool and an operation result to the record of the workflow (step S808).

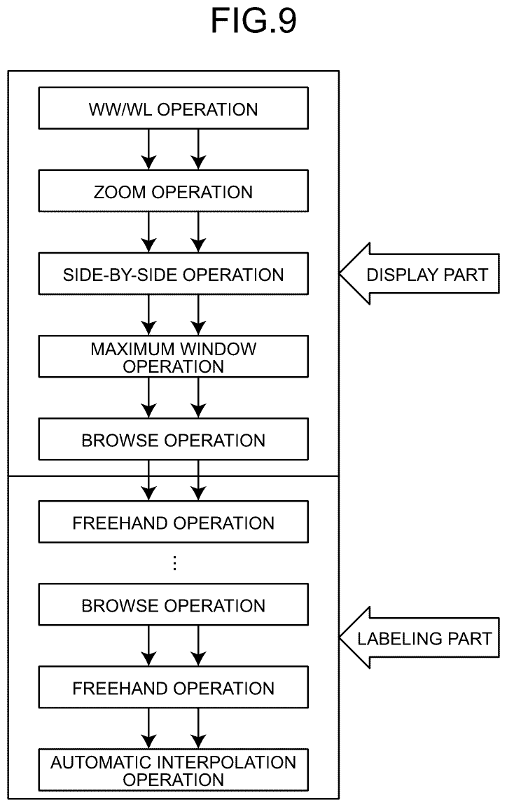

As explained herein, in the present embodiment, it is possible to form a workflow record as illustrated in FIG. 9, for example, by recording the operation tools one by one. FIG. 9 illustrates an exemplary configuration of the workflow. The workflow illustrated in FIG. 9 can roughly be divided into two parts, namely, a display part indicating steps of adjusting a display state and a labeling part indicating steps of performing labeling processes by using labeling tools.

The display part is primarily represented by operations to determine the position of a medical image displayed in the display on a display. A Window Width/Window Level (WW/WL) operation, a zoom operation, a side-by-side operation, a maximum window operation, and a browse operation are included. These operations correspond to a WW/WL tool, a zoom tool, a side-by-side tool, a maximum window tool, and a display slice determining tool, respectively. Because there is a possibility that the window position determining process on the display may involve frequently performing various types of operations, a plurality of operations using mutually the same tool are put together in the display part of the workflow, so as to record only a corresponding final operation result.

In contrast, the labeling part is primarily represented by steps of performing segmentation labeling on the medical image by using labeling tools. In FIG. 9, a freehand operation, a browse operation, and an automatic interpolation operation are included. These operations correspond to a freehand tool operation, a slice image switching selecting operation, and using an automatic interpolation tool, respectively.

In the example in FIG. 9, both the display part and the labeling part each have a record of a browse operation. The browse operation in the display part is an operation of finally determining a view to be displayed and thus belongs to the display tools. In contrast, the browse operation in the labeling part is an operation of switching to a slice image to be processed next, by switching between displayed views and thus belongs to the labeling tools. The tools used in these browse operations may be the same tool. In other words, there are one or more tools that belong to both the display tools and the labeling tools.

Further, in FIG. 9, because it is necessary to perform a browse operation and a freehand operation on each slice image, there are a plurality of sets each made up of a browse operation and a freehand operation. The labeling tool such as the freehand tool used in the freehand operation may be a labeling tool selected by the user from the tool set generated by the tool set generating function 50 or may be a labeling tool that is separately loaded by the user and used.

The configuration of the workflow in FIG. 9 is merely an example. Needless to say, it is acceptable to record the workflow by using other structures and/or rules. For example, changes in the image display and the tools used in accordance therewith may sequentially be recorded. As another example, actions considered erroneous operations may be set in advance so as to omit recording these actions. Naturally, the format of the workflow in FIG. 9 is not limited, either.

Further, returning to the description of FIG. 1, the labeling assisting function 70 is capable of assisting the labeling task by using the tool set and the workflow. For example, when the searching function 30 has found a usable tool set in the search, the labeling assisting function 70 is configured to output the usable tool set corresponding to the labeling task, as a candidate labeling tool. Further, the labeling assisting function 70 is configured to assist the medical image labeling process, on the basis of the workflow corresponding to the labeling task and to cause at least a part of the medical image labeling steps to conform to the abovementioned workflow. The labeling assisting function 70 is an example of a “labeling assisting unit”.

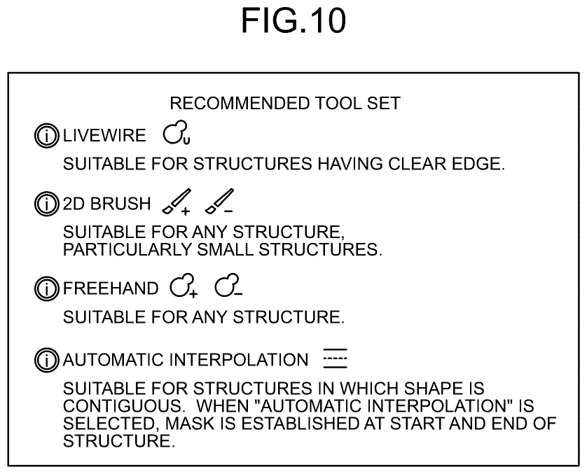

For example, let us discuss an example in which, after the labeling task is received, the searching function 30 has found in a search that there is a usable tool set corresponding to the received labeling task. In that situation, the labeling assisting function 70 is configured to present the usable tool set to the user via an output mechanism such as a speaker, a screen, or the like, so as to recommend that the user use the usable tool set during the labeling steps. For example, via a human machine interface, the labeling assisting function 70 is configured to present, to the user, a recommendation tool panel as illustrated in FIG. 10. In this situation, the labeling assisting function 70 is configured to list, in the recommendation tool panel, the labeling tools included in the tool set such as those of magnet selection, a 2D brush, freehand, and an automatic interpolation, and/or the like and annotation parts of relevant tools, so that the user is able to directly click on a corresponding tool so as to invoke and use the application of any of the abovementioned tools.

Let us discuss another example in which the searching function 30 has found in a search that there is an existing workflow corresponding to the received labeling task. In other words, let us assume that the searching function 30 has found the workflow that was previously generated and saved with respect to a labeling task of the same type as the received labeling task. In this situation, the labeling assisting function 70 is configured to assist execution of the labeling task by using the existing workflow.

More specifically, by referring to the existing workflow, the labeling assisting function 70 is capable of executing the received labeling task, according to the flow and the tools in the existing workflow. Further, the labeling assisting function 70 may be configured to notify the user of the existing workflow and to allow the user to decide whether or not a flow that is the same or partially the same as the existing workflow is to be adopted.

For example, let us assume that the existing workflow is a workflow including the two parts, namely, the display part and the labeling part, as illustrated in FIG. 9. In this situation, to begin with, at the time of invoking and displaying the data of the medical image, the labeling assisting function 70 is configured to at first apply the display part of the workflow. Subsequently, the labeling assisting function 70 is configured to extract information about the window width/window level, a zoom scale, a maximum window, and/or the like serving as a final operation result of the display part and to further automatically adjust the display state of the image in accordance with the information of these parameters. As explained herein, the workflow includes the display part indicating the steps of adjusting the display state, and the labeling assisting function 70 is configured, when the searching function 30 has found the existing workflow in the search, to adjust the display state of the displayed medical image in accordance with the final display result of the display part in the existing workflow.

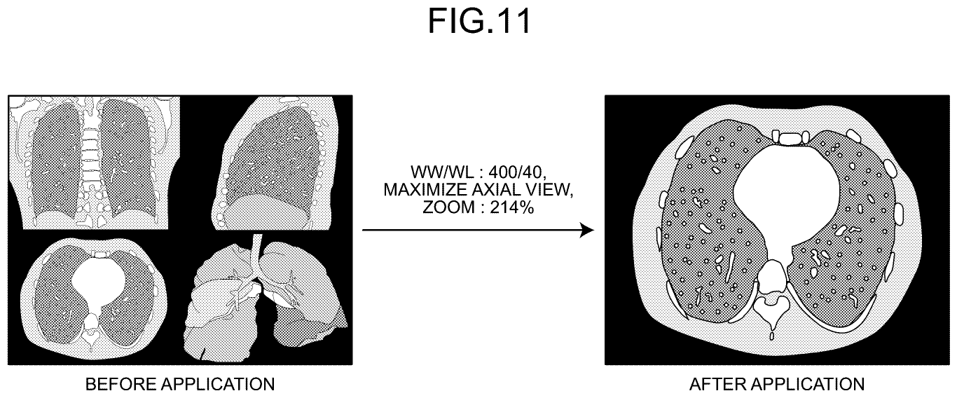

FIG. 11 is a schematic drawing illustrating a display example of when the display part of the workflow is applied. As illustrated in FIG. 11, the screen before the application is an initial screen displayed at the time of initially invoking the data on the screen after the labeling task was received. In the example in FIG. 11, after the processes are performed on the screen before the application so as to set the window width/window level to 400/40, to have a maximized axial view, and to zoom to 214%, the display screen automatically changes to the image after the application. The image after the application is more effective for the labeling process performed by the user, is able to save the user the trouble of adjusting the image state, and is thus able to enhance efficiency of the labeling process.

Further, the labeling assisting function 70 may be configured to form and display a flowchart for performing a labeling process by using labeling tools, in accordance with the details in the labeling part of the existing workflow. More specifically, the workflow includes the labeling part indicating the steps of performing labeling processes by using the labeling tools, so that when the searching function 30 has found an existing workflow in the search, the labeling assisting function 70 may form and display the flowchart for performing the labeling processes by using the labeling tools, according to the labeling part of the existing workflow.

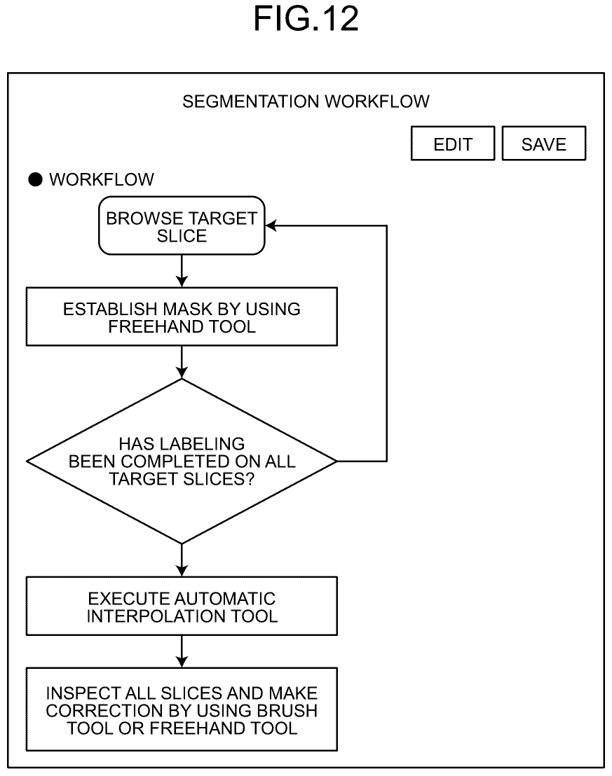

FIG. 12 is a schematic drawing illustrating a display example of when the labeling part of the workflow is applied. For example, a screen (the display 203) of the medical image processing apparatus 100 (In the present example, the medical image processing apparatus 100 may be a user terminal) displays a segmentation workflow as illustrated in FIG. 12, so as to guide the steps in the segmentation labeling process to be performed by the user. As indicated in the segmentation workflow, the user is able to label each slice image by using the freehand tool, to subsequently have an interpolation performed automatically by executing the automatic interpolation tool, and to finally make a correction by using either the brush tool or the freehand tool. The user may perform the labeling process by selecting a partial flow in the segmentation workflow and may omit the final correction step, for example. Also, the user may perform the labeling process by selecting one or more labeling tools from the tool set found in the search by the searching function 30, without following the rules in the segmentation workflow.

When having found in the search both a usable tool set and an existing workflow with respect to a single labeling task, the searching function 30 may recommend both of the two to the user or may recommend the usable tool set only if no workflow is present.

Further, when the tool set generating function 50 has generated a tool set, the generated tool set may be used for a later labeling process in the same labeling task or may be saved so as to be used for a different labeling task.

Furthermore, the medical image processing apparatus 100 may be configured to save the generated tool set or workflow as a product, which is to be transmitted to another apparatus for use therein. In that situation, the labeling assisting function 70 may be omitted.

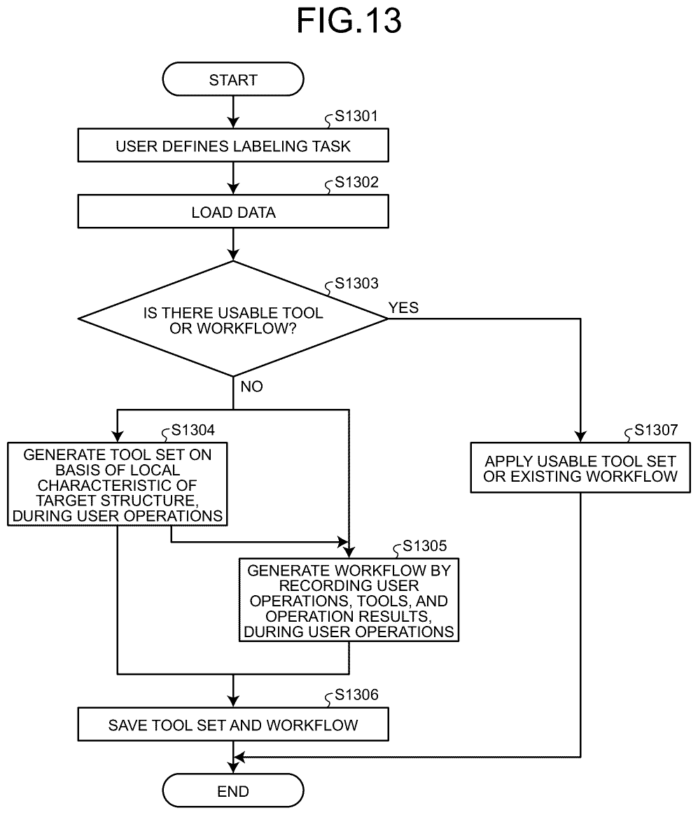

Next, an overall process performed by the medical image processing apparatus 100 according to the first embodiment will be explained. FIG. 13 is a flowchart illustrating a procedure in a process (a medical image processing method) performed by the medical image processing apparatus 100 according to the first embodiment.

To begin with, the user defines a labeling task via a human machine interface (step S1301). Accordingly, the receiving function 20 receives the definition of the labeling task and starts receiving the steps in the labeling process. Subsequently, at step S1302, the medical image data is loaded and displayed. In that situation, on the basis of the labeling task received by the receiving function 20, the searching function 30 searches for a usable tool set and an existing workflow corresponding to the obtained medical image labeling task (step S1303).

When the searching function 30 found a usable tool set or a workflow (step S1303: Yes), the labeling assisting function 70 assists the medical image labeling process by applying the usable tool set or the existing workflow (step S1307).

On the contrary, when the searching function 30 found neither a usable tool set nor a workflow (step S1303: No), the tool set generating function 50 generates, at step S1304, a usable tool set corresponding to the medical image labeling task, on the basis of a local characteristic of a partially-labeled target structure, during the user operations. After that, at step S1305, the workflow generating function 60 generates a workflow indicating medical image labeling steps, by recording user operations, tools, and operation results during the user operations. In this situation, the tool set generated at step S1304 may be applied during the subsequent labeling steps. In that situation, the workflow generating process may include an operation performed by using a tool selected by the user from the tool set generated at step S1304. Subsequently, at step S1306, the generated tool set and workflow are saved, and the labeling process is thus ended.

In conventional labeling work, all the usable tools are offered to a user, and information about types of segmentation for which the tools are suitable or the like is written in a manual. However, because there is no tool set for specific segmentation work, the user would need to look for and select a tool set. In contrast, in the present embodiment, the tool set is generated in correspondence with the characteristic of the image. In this regard, the relevance between image characteristics and tools may be set in advance, so that a tool set can be selected on the basis of whether a characteristic satisfies a specific condition. Consequently, in the present embodiment, it is possible to recommend a more appropriate tool. In addition, it is possible to shorten the labeling period of the user and to thus enhance the efficiency of the labeling process.

Further, according to conventional techniques, users perform processes such as segmentation on images by artificially applying tools. However, because artificial operations are voluntary, there is a possibility that the operations may follow a workflow familiar to everyone, instead of having a fixed pattern. In contrast, in the present embodiment, the workflow is generated by recording the steps in the labeling steps. As a result, to the same type of image labeling process in the future, it is possible to apply the generated workflow without any modification. It is therefore possible to enhance the efficiency of the labeling process.

In particular, in the present embodiment, because the workflow is generated, and the initial display of the image is automatically arranged by using the existing workflow, it is possible to significantly shorten the operation time of the user, when a large volume of data labeling process is performed on a certain labeling target. For instance, a conventional example of an image state adjusting process requires at least four steps, namely, (I) loading data, followed by (II) adjusting the window width/window level, (III) adjusting the view to a maximized axial view, and (IV) carrying out the zoom. In contrast, when the present embodiment is applied to this example, simply performing the step “(I) loading data” is able to achieve the display state conventionally achieved by performing (I) to (IV). It is therefore possible to save the time for performing steps (II) to (IV). Consequently, according to the present embodiment, it is possible to enhance the efficiency of the labeling process performed by the user.

Furthermore, according to the first embodiment, the appropriate tool set and workflow are automatically recommended on the basis of the labeling task defined by the user. There is no need to guide the user operations with a fixed flow. It is therefore possible to make the assistance for the labeling work more flexible. It is therefore similarly possible to enhance the efficiency of the labeling process performed by the user.

### Modification Examples of First Embodiment

The present disclosure is not limited to the configurations in the first embodiment described above and may be modified in various manners.

For example, in the configuration of the first embodiment, the medical image processing apparatus 100 is configured, by employing the searching function 30, to search for a usable tool set or an existing workflow. Let us discuss a situation in which, for example, neither an existing usable tool set nor a workflow is present. In that situation, the medical image processing apparatus 100 generates and saves a tool set or a workflow, which is to be offered to another apparatus as a product, so that the other apparatus applies the tool set or the workflow. In that situation, the searching function 30 and the labeling assisting function 70 may be omitted. When the searching function 30 and the labeling assisting function 70 are omitted, steps S1303 and S1307 are omitted from the flowchart in FIG. 13.

Further, the tool set using and generating processes may be independent from the workflow using and generating processes. In other words, the workflow does not need to have the step of referencing a tool set.

In this manner, for example, the medical image processing apparatus 100 may have only the configuration related to the tool set generating process, while the analyzing function 40 and the workflow generating function 60 and omitted. In that situation, step S1305 is omitted from the flowchart in FIG. 13.

In yet another example, the medical image processing apparatus 100 may have only the configuration related to the workflow generating process, while the workflow generating function 60 is omitted. In that situation, step S1304 is omitted from the flowchart in FIG. 13.

### Second Embodiment

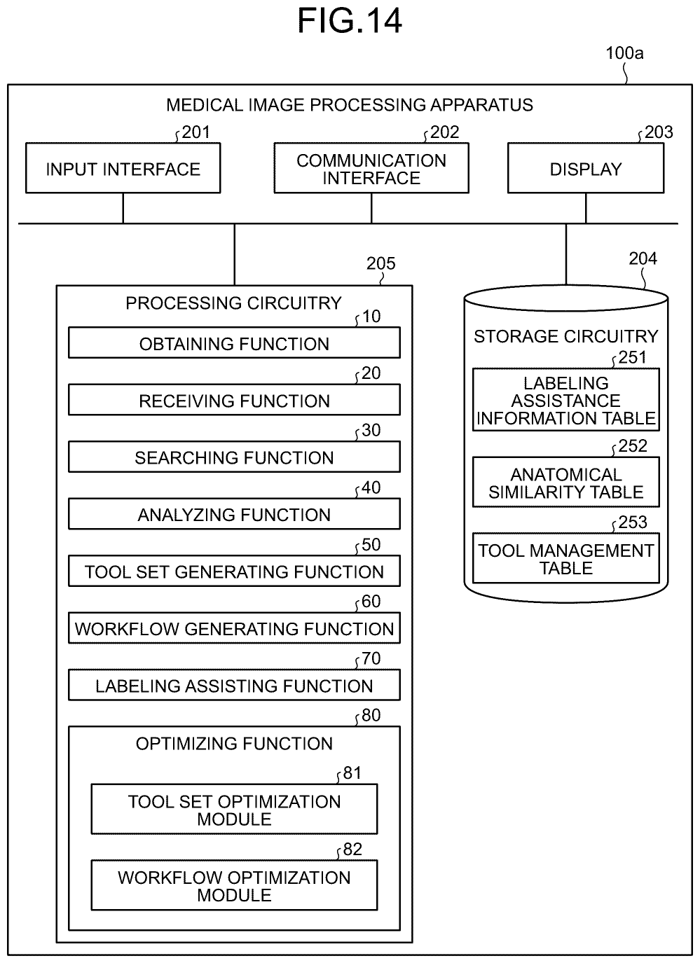

A second embodiment will be explained, with reference to FIGS. 14 to 20. A medical image processing apparatus 100a according to the second embodiment is primarily different from the first embodiment for further including an optimizing function 80. In the following sections, differences will primarily be explained. Some of the elements that are the same as or similar to those in the first embodiment will be referred to by using the same reference characters, and duplicate explanations will be omitted as appropriate.

FIG. 14 is a block diagram illustrating a functional configuration of the medical image processing apparatus 100a according to the second embodiment.

As illustrated in FIG. 14, the processing circuitry 205 of the medical image processing apparatus 100a includes the obtaining function 10, the receiving function 20, the searching function 30, the analyzing function 40, the tool set generating function 50, the workflow generating function 60, the labeling assisting function 70, and the optimizing function 80.

In this situation, processing functions executed by the constituent elements of the processing circuitry 205 illustrated in FIG. 14, namely, the obtaining function 10, the receiving function 20, the searching function 30, the analyzing function 40, the tool set generating function 50, the workflow generating function 60, the labeling assisting function 70, and the optimizing function 80, are recorded in the storage circuitry 204 of the medical image processing apparatus 100a in the form of computer-executable programs, for example.

Next, details of processes performed by the obtaining function 10, the receiving function 20, the searching function 30, the analyzing function 40, the tool set generating function 50, the workflow generating function 60, the labeling assisting function 70, and the optimizing function 80 executed by the processing circuitry 205 will be explained.

The obtaining function 10 is configured to obtain a medical image that was acquired by scanning the patient and needs to be labeled, from an image acquiring apparatus. The medical image is subject to a labeling process performed by any of various types of labeling tools. In other words, the obtaining function 10 is configured to obtain the medical image subject to the labeling process. The obtaining function 10 is an example of an “obtaining unit”.

The receiving function 20 is configured to receive labeling steps in a labeling task performed on the medical image obtained by the obtaining function 10. The receiving function 20 is an example of a “receiving unit”.

On the basis of the labeling task received by the receiving function 20, the searching function 30 is configured to conduct a search for a usable tool set and an existing workflow corresponding to the obtained medical image labeling task. The searching function 30 is an example of a “searching unit”.

While the receiving function 20 is receiving the labeling steps in the labeling task, the analyzing function 40 is configured to analyze a local characteristic of a target structure serving as a labeling target in the analyzed medical image. Accordingly, the tool set generating function 50 is configured to generate a usable tool set corresponding to the medical image labeling task, on the basis of the local characteristic analyzed by the analyzing function 40. The analyzing function 40 is an example of an “analyzing unit”. The tool set generating function 50 is an example of a “tool set generating unit”.

The workflow generating function 60 is configured to record the labeling steps in the labeling task received by the receiving function 20 and to generate a workflow indicating the medical image labeling steps. The workflow generating function 60 is an example of a “workflow generating unit”.

Further, the labeling assisting function 70 is capable of assisting the labeling task by using the tool set and the workflow. For example, when the searching function 30 has found a usable tool set in the search, the labeling assisting function 70 is configured to output the usable tool set corresponding to the labeling task, as a candidate labeling tool. Further, the labeling assisting function 70 is configured to assist the medical image labeling process, on the basis of the workflow corresponding to the labeling task and to cause at least a part of the medical image labeling steps conform to the workflow. The labeling assisting function 70 is an example of a “labeling assisting unit”.

Because the configurations and the operations of the obtaining function 10, the receiving function 20, the searching function 30, the analyzing function 40, the tool set generating function 50, the workflow generating function 60, and the labeling assisting function 70 according to the second embodiment are substantially the same as those in the first embodiment, detailed explanations thereof will be omitted in the present embodiment.

Further, the optimizing function 80 includes a tool set optimization module 81 for optimizing a tool set and a workflow optimization module 82 for optimizing a workflow.

In this situation, after the receiving function 20 finishes receiving the labeling steps in the labeling task, the analyzing function 40 is configured to analyze a global characteristic of the target structure, so that the tool set optimization module 81 further optimizes the existing usable tool set on the basis of the global characteristic of the target structure. For example, after the labeling task is completed, the tool set optimization module 81 is configured to evaluate a candidate tool that is not included in the usable tool set, on the basis of the global characteristic of the target structure in a labeled result of the labeling task. After that, the tool set optimization module 81 is configured to optimize the usable tool set, by adding the tool determined to be suitable for the completed labeling task as a result of evaluating the candidate tool, to the usable tool set corresponding to the labeling task. The tool set optimization module 81 is an example of a “tool set optimizing unit”.

In the following sections, an example of a labeling task to perform segmentation on a lung part will be explained. In the following example, it is assumed that neither the tool set used in the segmentation labeling task nor the tool set generated in the steps of the segmentation labeling task includes an automatic interpolation tool.

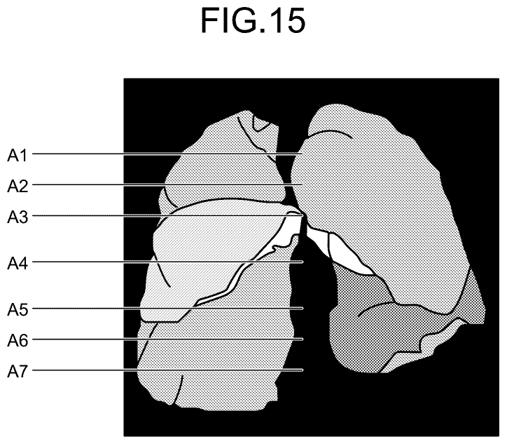

Let us assume that, as a result of the segmentation labeling task, a three-dimensional lung part image serving as a target structure is obtained as illustrated in FIG. 15. In this situation, the tool set optimization module 81 is configured to extract a shape characteristic from the target structure and to judge whether or not an automatic interpolation tool is suitable for the lung part segmentation labeling task, on the basis of the shape characteristic. When the automatic interpolation tool is selected at the time of performing the labeling task, the user performs segmentation on a number of slice images such as slice images A1 to A7 (key slices) in FIG. 15, for example, and subsequently generates a segmentation result for other parts by using the automatic interpolation tool.

For example, because the lung part has a relatively regular shape, it is possible to divide the lung part image into a plurality of zones. Thus, on the lung part image, after the user manually performs the segmentation on the slice images A1 to A7, it is possible to generate other zones by using the automatic interpolation tool. For example, in FIG. 15, on the image of the right lung in the lung part, after the user manually performs the segmentation on the slice images A1 to A7, it is possible to generate images of the other zones of the right lung, by interpolating the zones between the slice images A1 to A7 with the use of the automatic interpolation tool. As another example, in FIG. 15, on the image of the right lung in the lung part, after the user manually performs the segmentation on the slice images A1 to A7, it is possible to generate images of the left lung, by interpolating the left lung while using the slice images A1 to A7 with the use of the automatic interpolation tool.

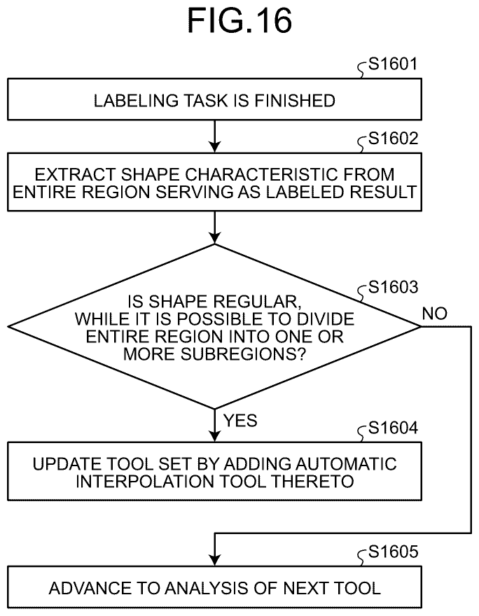

FIG. 16 is a flowchart for judging applicability of the automatic interpolation tool in the tool set optimizing process. As illustrated in FIG. 16, after the labeling task is finished (step S1601), the tool set optimization module 81 extracts a shape characteristic from the entire region serving as a labeled result (step S1602). Subsequently, at step S1603, on the basis of the extracted shape characteristic, the tool set optimization module 81 judges whether or not the labeled result is regular, while it is possible to divide the labeled result into one or more subregions. At this time, let us discuss a situation where the tool set optimization module 81 determines that the labeled result is regular and that it is possible to divide the labeled result into a plurality of subregions, for example, possible to divide the lung part image into two subregions on the left and the right as illustrated in FIG. 15 (step S1603: Yes). In that situation, the tool set optimization module 81 updates the already-generated tool set by adding the automatic interpolation tool thereto (step S1604). On the contrary, let us discuss another situation where the tool set optimization module 81 determines that the labeled result is irregular or that it is not possible to divide the labeled result into one or more subregions (step S1603: No). In that situation, the tool set optimization module 81 judges a next candidate tool (step S1605).

As explained herein, in the present embodiment, it is possible to update the tool set by judging a plurality of candidate tools. Selected as the candidate tools may be the tools for which the applicability was judged when the tool set generating function 50 generated the tool set or may be tools for which the applicability was not judged when the tool set generating function 50 generated the tool set.

Alternatively, the analyzing function 40 may analyze a global characteristic of the target structure in the labeling task and supply an analysis result to the tool set optimization module 81, so that the tool set optimization module 81 judges whether or not the tool needs to be added to the tool set on the basis of the global characteristic.

Returning to the description of FIG. 14, the workflow optimization module 82 is configured to correct the existing workflow so as to optimize the workflow. More specifically, the analyzing function 40 is configured to analyze a global characteristic of the labeled result of the labeling task received by the receiving function 20, so that the workflow optimization module 82 corrects the existing workflow so as to optimize the workflow. The workflow optimization module 82 is an example of a “workflow optimizing unit”.

The method used by the workflow optimization module 82 to perform the optimization is not particularly limited. The workflow optimization module 82 may correct parameters used in the workflow, on the basis of the global characteristic of the labeled result analyzed by the analyzing function 40. For example, the workflow optimization module 82 may correct a display parameter or a labeling parameter in the workflow, on the basis of the characteristic of the labeled result or a reference level such as an industrial standard in the relevant field.

Further, in the present embodiment, the precision level of the labeling process may further be enhanced by adding a new operation to the workflow, e.g., adding an operation to use a new labeling tool. Alternatively, for example, with respect to a part of the operations in the workflow, it is also acceptable to use an operation of a new tool that is more accurate and advanced, in place of an original operation.

As an example of the workflow optimization, for instance, on the basis of the characteristic analysis on the labeled result, the workflow optimization module 82 may further perform optimization on the display position determining parameters resulting from using the tools in the display part of the workflow.

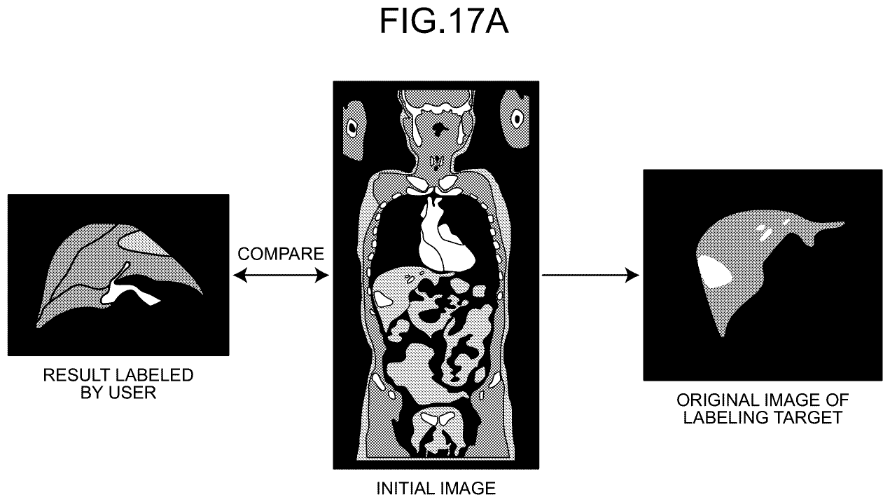

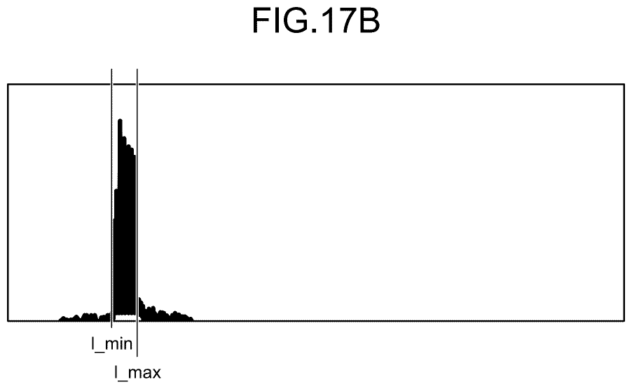

FIGS. 17A and 17B are examples for explaining the optimization on the parameters in the display part. With reference to FIGS. 17A and 17B, an example of a labeling task to segment the liver will be explained.

The workflow optimization module 82 is configured, as illustrated in FIG. 17A, to determine the region of the liver within the original image (i.e., the original image serving as a labeling target in FIG. 17A), by comparing a labeled result of the liver labeled by the user, with the original image of the medical image obtained by the obtaining function 10 as an image subject to the labeling process. In this situation, when the workflow optimization module 82 displays the region of the liver within the original image by using optimal window width/window level values, for example, as a display state in the display part of the workflow, it will be more effective for the labeling process performed by the user.

Accordingly, the workflow optimization module 82 is configured to analyze a gradation histogram as illustrated in FIG. 17B with respect to the entire region of the labeling target. For example, the workflow optimization module 82 is configured to obtain a pixel minimum value I_min and a pixel maximum value I_max in the gradation histogram and to calculate window width/window level values to be optimal window width/window level values, by using Expression (1) presented below, for example. After that, the workflow optimization module 82 is configured to switch the window width/window level values in the existing workflow into the calculated window width/window level values.

left=I_min×slope+intercept

right=I_max×slope+intercept

WINDOW WIDTH: WW=right−left

WINDOW LEVEL: WL=(right+left)/2 (1)

In Expression (1), “left” and “right” are variables, while “slope” denotes a slope, and “intercept” denotes an intercept.

As a result, the workflow after the update is capable of realizing a display that puts together the regions where a labeling target is present. It is therefore more effective for the labeling process.

Further, while the workflow is structured with a plurality of operation steps, the workflow optimization module 82 may add a new operation step to the workflow, on the basis of the global characteristic of the labeled result analyzed by the analyzing function 40. For example, with respect to the labeling part of the workflow, the workflow optimization module 82 may select a number of labeling tools as candidate tools and judge whether or not the additional use of each of the candidate tools is suitable, on the basis of the global characteristic of the labeling target. Upon determining that the additional use of any of the candidate tools is appropriate, the workflow optimization module 82 may add the operation on the candidate tool to the workflow.

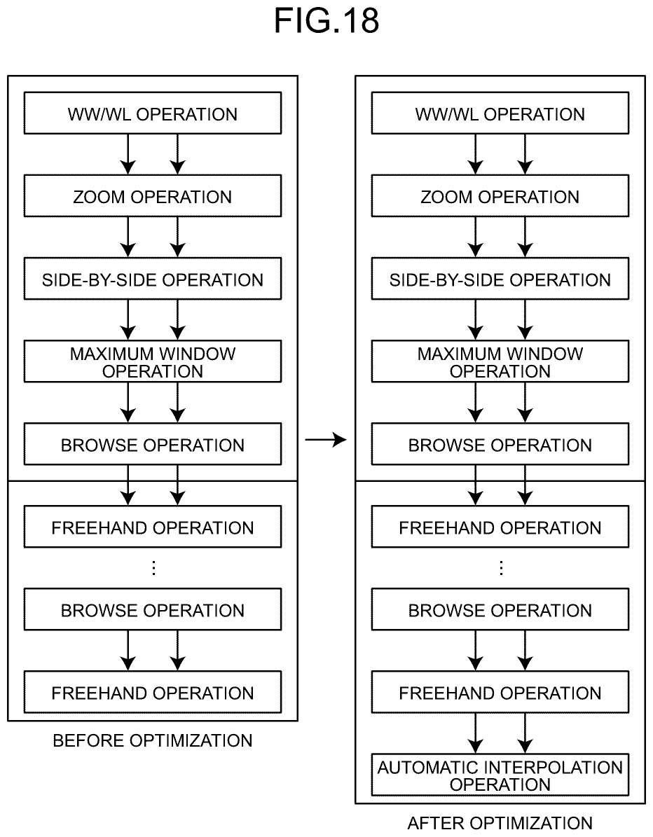

For instance, let us discuss an example in which the original workflow is structured with the plurality of operation steps illustrated in FIG. 18. In that situation, the workflow optimization module 82 is configured to perform the judging process by using the automatic interpolation tool as a candidate tool and to extract a shape characteristic from the labeled result of the labeling task in the workflow having no automatic interpolation tool recorded. In this situation, the workflow optimization module 82 is configured to judge whether or not the shape is regular, while it is possible to divide the entire region serving as the labeled result into one or more subregions. In this situation, upon determining that the shape is regular and that it is possible to divide the entire region serving as the labeled result into one or more subregions, the workflow optimization module 82 determines that the automatic interpolation tool is also suitable for the use. In that situation, the workflow optimization module 82 inserts an automatic interpolation operation at the end of the pre-optimization workflow illustrated on the left-hand side of FIG. 18, to obtain a post-optimization workflow as illustrated on the right-hand side of FIG. 18.

Further, while the workflow is structured with a plurality of operation steps, the workflow optimization module 82 may replace a part of the operation steps in the workflow with one or more new operation steps. For example, the workflow optimization module 82 may optimize the workflow, by replacing an operation step having an equivalent or similar function in the workflow and replacing a certain operation step with a more capable operation step.

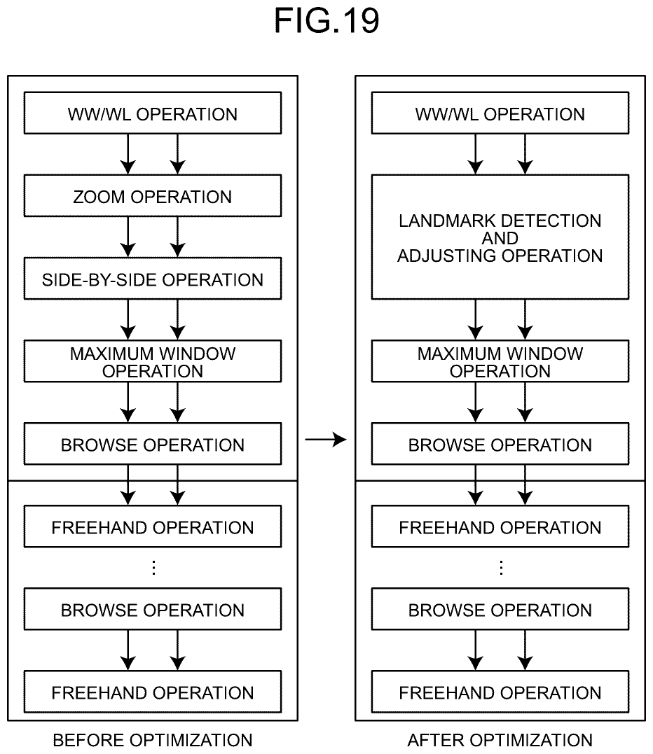

More specifically, the workflow may include an operation step for adjusting a display range, so that the workflow optimization module 82 replaces the operation step for adjusting the display range, with an operation step for identifying a display range by detecting a landmark. For example, when an original workflow has a configuration as illustrated in FIG. 19, the workflow optimization module 82 may replace a zoom operation and a side-by-side operation that were originally included in the workflow, with a landmark detection and an adjusting operation. More specifically, the workflow optimization module 82 obtains a post-optimization workflow as illustrated on the right-hand side of FIG. 19, by replacing the step at which the user manually adjusts a display state by using a zoom tool and a side-by-side tool indicated in the pre-optimization workflow on the left-hand side of FIG. 19, with the step at which a landmark tool automatically detects a landmark from the medical image so that a display parameter is automatically calculated on the basis of the landmark.

As for specifics methods for using the landmark tool, it is possible to refer to any of various use methods that are already mature in conventional techniques. For example, in the present embodiment, it is possible to identify parameter values for zooming and side-by-side operations to be applied to the original image, by detecting a plurality of landmarks from the medical image, so as to set, on the basis of the landmarks, a range frame having the smallest area possible while including all the landmarks, and further calculating view center position information on the basis of coordinate information and center position information of the range frame. In this manner, in the present embodiment, when the workflow is applied, it is possible to automatically calculate the appropriate parameter values for the zooming and the side-by-side operations, so as to make the automatic adjustment to obtain the appropriate view.

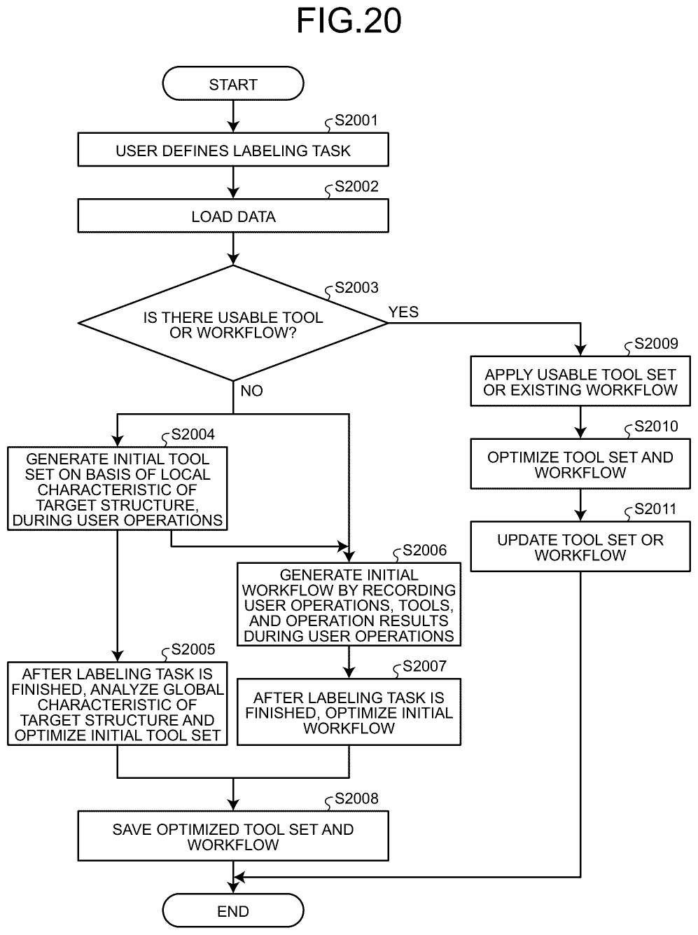

Next, an overall process performed by the medical image processing apparatus 100a according to the second embodiment will be explained. FIG. 20 is a flowchart illustrating a procedure in a process (a medical image processing method) performed by the medical image processing apparatus 100a according to the second embodiment.

To begin with, the user defines a labeling task via a human machine interface (step S2001). Accordingly, the receiving function 20 receives the definition of the labeling task and starts receiving the steps in the labeling process. Subsequently, at step S2002, the medical image data is loaded and displayed. The searching function 30 searches for a usable tool set and an existing workflow corresponding to the obtained medical image labeling task, on the basis of the labeling task received by the receiving function 20 (step S2003).

When the searching function 30 found a usable tool set or a workflow in the search (step S2003: Yes), the labeling assisting function 70 assists the medical image labeling process by applying the usable tool set or the existing workflow (step S2009). Further, during the applying step or after the application is finished, the tool set optimization module 81 optimizes the tool set on the basis of a characteristic of the labeling target, and the workflow optimization module 82 optimizes the existing workflow (step S2010). As a result, the tool set or the workflow is updated (step S2011).

On the contrary, when the searching function 30 found neither a usable tool set nor a workflow in the search (step S2003: No), the tool set generating function 50 generates, at step S2004, an initial tool set corresponding to the medical image labeling task, on the basis of a local characteristic of a partially-labeled target structure, during the user operations. After that, at step S2005, after the labeling task is finished, the tool set optimization module 81 analyzes a global characteristic of the target structure serving as the labeled result and optimizes the initial tool set on the basis of the global characteristic.

Further, in parallel to step S2004, at step S2006, the workflow generating function 60 generates an initial workflow indicating the medical image labeling steps, by recording user operations, tools, and operation results during the user operations. In this situation, the tool set generated at step S2004 may be applied during the subsequent labeling steps. While a workflow is generated, the operations may include an operation performed by using a tool selected by the user from the tool set generated at step S2004. Subsequently, at step S2007, after the labeling task is finished, the workflow optimization module 82 optimizes the initial workflow and saves the optimized tool set and workflow, and the labeling process is thus ended (step S2008) According to the second embodiment, it is possible to achieve advantageous effects similar to those of the first embodiment. Further, even after the labeling task is finished, it is possible to optimize the tool set by using the labeled result. Consequently, it is possible to recommend a more appropriate tool, at a future time when a similar labeling task is executed by using the post-update tool set. It is therefore possible to further enhance the efficiency of the labeling process.

Further, according to the second embodiment, it is possible to optimize the workflow and to thus enhance the efficiency of the labeling process performed by the user. For example, by effectively using the automatic interpolation tool, it is possible to significantly shorten the labeling time of the user and to thus enhance the efficiency of the labeling process. Further, because it is possible to automatically optimize the tool set and the workflow, additional wizards become unnecessary.

The constituent elements of the medical image processing apparatuses in the above embodiments are functional and conceptual. Thus, it is not necessarily required to physically configure the constituent elements as indicated in the drawings. In other words, specific modes of distribution and integration of the medical image processing apparatuses are not limited to those illustrated in the drawings. It is acceptable to functionally or physically distribute or integrate all or a part of the apparatuses in any arbitrary units, depending on various loads and the status of use. Further, all or an arbitrary part of the processes and functions performed by the medical image processing apparatuses may be realized by a CPU and a program analyzed and executed by the CPU or may be realized as hardware using wired logic.

Further, it is possible to realize any of the medical image processing apparatuses explained in the above embodiments, by causing a computer such as a personal computer or a workstation to execute a program prepared in advance. The program may be distributed via a network such as the Internet. Further, the program may further be executed, as being recorded on a non-transitory computer-readable recording medium such as a hard disk, a flexible disk (FD), a Compact Disk Read-Only Memory (CD-ROM), a Magneto Optical (MO) disk, a Digital Versatile Disk (DVD), or the like and being read by a computer from the recording medium.

Furthermore, the tool set or the workflow generated by any of the medical image processing apparatuses may be recorded and transported on a storage medium or the like as a product, so that the product is used as being loaded into another labeling apparatus.

According to at least one aspect of the embodiments described above, it is possible to enhance the efficiency of the labeling process performed by the user.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising processing circuitry configured:
to obtain a medical image subject to a labeling process;
to receive a labeling step in a labeling task performed on the medical image;
to analyze, while the labeling step in the labeling task is received, a local characteristic of a target structure serving as a labeling target in the medical image; and
to generate a usable tool set corresponding to the labeling task performed on the medical image, on a basis of the local characteristic.

2. The medical image processing apparatus according to claim 1, wherein the usable tool set is a tool set including a plurality of labeling tools.

3. The medical image processing apparatus according to claim 1, wherein
after having finished receiving the labeling step in the labeling task, the processing circuitry is configured to analyze a global characteristic of the target structure, and
the processing circuitry is configured to optimize the usable tool set being an existing usable tool set, on a basis of the global characteristic of the target structure.

4. The medical image processing apparatus according to claim 1, wherein the processing circuitry is configured to record the received labeling step in the labeling task and to generate a workflow indicating the labeling step performed on the medical image corresponding to the labeling task performed on the medical image.

5. The medical image processing apparatus according to claim 1, wherein the processing circuitry is configured to conduct a search to determine whether or not a usable tool set corresponding to the labeling task performed on the medical image is present.

6. The medical image processing apparatus according to claim 5, wherein, upon finding the usable tool set in the search, the processing circuitry is configured to output the usable tool set as a candidate labeling tool.

7. A medical image processing apparatus comprising processing circuitry configured:
to obtain a medical image subject to a labeling process;
to receive a labeling step in a labeling task performed on the medical image; and
to record the received labeling step in the labeling task and to generate a workflow indicating the labeling step performed on the medical image.

8. The medical image processing apparatus according to claim 7, wherein the processing circuitry is configured to conduct a search to determine whether or not an existing workflow corresponding to the labeling task performed on the medical image is present.

9. The medical image processing apparatus according to claim 7, wherein the processing circuitry is configured to assist the labeling process performed on the medical image on a basis of the workflow and to cause at least a part of the labeling step performed on the medical image to conform to the workflow.

10. The medical image processing apparatus according to claim 7, wherein the workflow includes a display part indicating a step for adjusting a display state and a labeling part indicating a step for performing the labeling process by using a labeling tool.

11. The medical image processing apparatus according to claim 8, wherein
the workflow includes a display part indicating a step for adjusting a display state, and
upon finding the existing workflow in the search, the processing circuitry is configured to adjust a display state of the medical image being displayed, in accordance with a final display result in the display part of the existing workflow.

12. The medical image processing apparatus according to claim 8, wherein
the workflow includes a labeling part indicating a step for performing the labeling process by using a labeling tool, and
upon finding the existing workflow in the search, the processing circuitry is configured to form and display a flowchart for performing the labeling process by using the labeling tool, according to the labeling part of the existing workflow.

13. The medical image processing apparatus according to claim 7, wherein
the processing circuitry is configured to analyze a global characteristic of a labeled result from the received labeling task, and
the processing circuitry is configured to optimize the workflow by correcting the existing workflow.

14. The medical image processing apparatus according to claim 13, wherein the processing circuitry is configured to correct a parameter used in the workflow, on a basis of the analyzed global characteristic of the labeled result.

15. The medical image processing apparatus according to claim 13, wherein
the workflow is structured with a plurality of operation steps, and
the processing circuitry is configured to add a new operation step to the workflow, on a basis of the analyzed global characteristic of the labeled result.

16. The medical image processing apparatus according to claim 13, wherein
the workflow is structured with a plurality of operation steps, and
the processing circuitry is configured to replace a part of the operation steps in the workflow with one or more new operation steps.

17. The medical image processing apparatus according to claim 16, wherein
the workflow includes an operation step for adjusting a display range, and
the processing circuitry is configured to replace the operation step for adjusting the display range, with an operation step for identifying a display range by detecting a landmark.

18. A medical image processing method comprising:
an obtaining step of obtaining a medical image subject to a labeling process;
a receiving step of receiving a labeling step in a labeling task performed on the medical image;
an analyzing step of analyzing, while the labeling step in the labeling task is received at the receiving step, a local characteristic of a target structure serving as a labeling target in the medical image; and
a tool set generating step of generating a usable tool set corresponding to the labeling task performed on the medical image, on a basis of the local characteristic.

19. A medical image processing method comprising:
an obtaining step of obtaining a medical image subject to a labeling process;
a receiving step of receiving a labeling step in a labeling task performed on the medical image; and
a workflow generating step of recording the labeling step in the labeling task received at the receiving step and generating a workflow indicating the labeling step performed on the medical image.
